(19) United States
(12) Patent Application Publication
(10) Pub. No.: US 2021/0150712 A1
(43) Pub. Date: May 20, 2021

(54) IMAGE ANALYSIS DEVICE AND IMAGE ANALYSIS METHOD

(71) Applicant: OLYMPUS CORPORATION, Tokyo (JP)

(72) Inventor: Kenro OSAWA, Tokyo (JP)

(73) Assignee: OLYMPUS CORPORATION, Tokyo (JP)

(21) Appl. No.: 17/159,299

(22) Filed: Jan. 27, 2021

Related U.S. Application Data
(63) Continuation of application No. PCT/JP2018/028664, filed on Jul. 31, 2018.

(51) Int. Cl. G06T 7/00; G06K 9/46

(52) U.S. Cl. CPC G06T 7/0012; G06T 2207/10056; G06T 2207/30096; G06K 9/4604

(57) ABSTRACT

A first local region category calculation unit calculates a first local region category of an input image. An image category calculation unit calculates an image category from the first local region category. An image category output unit outputs the image category. A second local region category calculation unit calculates a second local region category of the input image. A local region category output unit outputs the second local region category.

### CROSS-REFERENCE TO RELATED APPLICATIONS

This application is based upon and claims the benefit of priority from International Application No. PCT/JP2018/028664, filed on Jul. 31, 2018, the entire contents of which is incorporated herein by reference.

### BACKGROUND

The present disclosure relates to a technology for analyzing an input image.

An image analysis technology is known that locally analyzes a subject image and determines whether each of a plurality of local regions in the image is an abnormal region or a normal region without anomalies (see Patent Document 1, Non-Patent Document 1). In this image analysis technology, it is determined whether the image as a whole is an abnormal image including abnormal regions or a normal image not including abnormal regions based on the analysis result for each local region. By identifying abnormal images in this way and presenting the identified abnormal images to the observer of the image, it is possible to support efficient observation by the observer.

An image analysis technology for categorizing each of a plurality of local regions in an image using a neural network has been proposed (see Non-Patent Document 2). In this image analysis technology, the probability that each of a plurality of categories corresponds to each local region is estimated, a category with the highest probability is identified, and the category is defined as a local region category. A parameter for estimating a local region category with high accuracy is acquired by preparing a large number of datasets of an input image and correct categories of multiple local regions into which the input image is divided and performing supervised learning of neural network parameters (weight and bias).
[Patent Document 1] Japanese Patent Application Publication No. 2010-203949
[Non-Patent Document 1] Yun Liu, Krishna Gadepalli, Mohammad Norouzi, George E. Dahl, Timo Kohlberger, Aleksey Boyko, Subhashini Venugopalan, Aleksei Timofeev, Philip Q. Nelson, Greg S. Corrado, Jason D. Hipp, Lily Peng, and Martin C. Stumpe, “Detecting Cancer Metastases on Gigapixel Pathology Images”, arXiv:1703.02442v2 [cs.CV] 8 Mar. 2017
[Non-Patent Document 2] Liang-Chieh Chen, George Papandreou, Iasonas Kokkinos, Kevin Murphy, Alan L. Yuille, “SEMANTIC IMAGE SEGMENTATION WITH DEEP CONVOLUTIONAL NETS AND FULLY CONNECTED CRFS”, arXiv:1412.7062v4 [cs.CV] 7 Jun. 2016

According to the above-mentioned determination method, if there is even a single local region classified as an abnormal region in the plurality of local regions in the image, the image is determined to be an abnormal image, and if there is not even a single local region classified as an abnormal region, the image is determined to be a normal image. When a pathologist makes a pathological diagnosis, if an image containing lesions can be presented as an abnormal image, the pathologist can make an efficient diagnosis.

In such an image analysis process, it cannot be allowed to erroneously determine an image containing lesions as a normal image because the lesions may be overlooked by a pathologist if erroneous determination is performed. Therefore, it is necessary to effectively learn a large number of pathological images and acquire the parameters (weight and bias) of the neural network with improved estimation accuracy of each category of local regions in advance. However, in reality, it is not easy to achieve 100% category estimation accuracy, and there can be a situation where the probability of a category indicating a lesion and the probability of a category indicating no lesion show almost the same value.

As a method for not erroneously determining an image containing lesions as a normal image, one possible option is to raise the probability of a category indicating lesions to be higher than a calculated value when determining local region categories. By performing the raising process, the category indicating lesions can be more easily identified as a category with the highest probability, and as a result, the possibility that images containing lesions are erroneously determined as normal images can be reduced.

However, the raising process inevitably increases the possibility of erroneously determining an image that does not contain lesions as an abnormal image. When an image containing no lesion is presented to a pathologist as an abnormal image, the pathologist will spend time desperately searching for a lesion that does not exist, which is time consuming. Therefore, erroneous determination of an image containing no lesion as an abnormal image cannot be allowed because the erroneous determination does not support the image diagnosis performed by the pathologist but rather hinders rapid image diagnosis. Therefore, a technology for calculating image categories with high accuracy is desired.

Outputting the category of a local region for a correctly determined abnormal image is useful for improving the observation efficiency of the observer. For example, the category of a local region may be output as a visualization processing result such as coloring the position of the local region classified as an abnormal region. In particular, when multiple lesions in very small regions are scattered in an image of the entire specimen captured at high resolution, marking of the abnormal (lesion) positions is considered to be effective diagnostic support.

### SUMMARY

In this background, one of exemplary purposes of an embodiment of the present disclosure is to provide a technology for analyzing an input image, calculating the image category with high accuracy, and outputting the category of a local region useful for the user's image observation.

An image analysis device according to one embodiment of the present disclosure includes: a first local region category calculation unit that calculates a first local region category of an input image; an image category calculation unit that calculates an image category from the first local region category; an image category output unit that outputs the image category; a second local region category calculation unit that calculates a second local region category of the input image; and a local region category output unit that outputs the second local region category.

Another embodiment of the present disclosure relates to an image analysis method. This method includes: calculating a first local region category of an input image; calculating an image category from the first local region category; outputting the image category; calculating a second local region category of the input image; and outputting the second local region category.

Optional combinations of the aforementioned constituting elements, and implementations of the disclosure in the form of methods, apparatuses, systems, or the like may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram showing the configuration of an image analysis system according to an embodiment;

FIG. 2 is a diagram showing an example of a pathological image;

FIG. 3 is a diagram showing an example of a pathological image containing abnormal regions;

FIG. 4 is a diagram showing the configuration of a CNN;

FIG. 5 is a diagram showing the configuration of an image analysis device according to the first exemplary embodiment;

FIG. 6 is a diagram showing an output example of a first local region category;

FIG. 7 is a diagram showing an output example of an image category and a second local region category;

FIG. 8 is a diagram showing the configuration of an image analysis device according to the second exemplary embodiment;

FIG. 9 is a diagram showing the configuration of an image analysis device according to the third exemplary embodiment;

FIG. 10 is a diagram showing the configuration of an image analysis device according to the fourth exemplary embodiment;

FIG. 11 is a diagram showing the configuration of an image analysis device according to the fifth exemplary embodiment;

FIG. 12 is a diagram showing the configuration of an image analysis device according to the sixth exemplary embodiment;

FIG. 13 is a diagram showing the configuration of an image analysis device according to the seventh exemplary embodiment; and

FIG. 14 is a diagram showing the configuration of an image analysis device according to the eighth exemplary embodiment.

### DETAILED DESCRIPTION

The disclosure will now be described by reference to the preferred embodiments. This does not intend to limit the scope of the present disclosure, but to exemplify the disclosure.

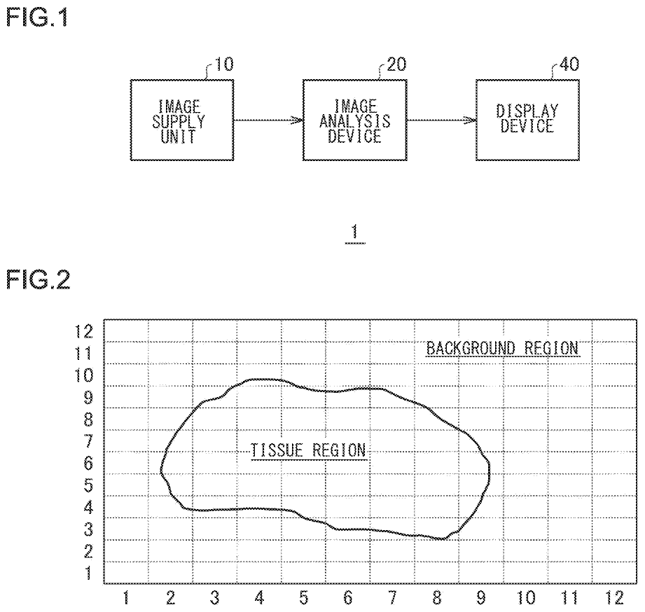

FIG. 1 shows the configuration of an image analysis system 1 according to an embodiment. The image analysis system 1 includes an image supply unit 10, an image analysis device 20, and a display device 40. The image supply unit 10 supplies an input image to the image analysis device 20. The image analysis device 20 divides the input image into a plurality of local regions, calculates a category for each of the plurality of local regions, and calculates a category for the entire image from the plurality of local region categories.

The input image according to the embodiment may be a pathological diagnosis image (pathological image) obtained by imaging a pathological specimen (tissue) on a slide glass in a magnified manner through a microscope. The pathological image is a color image composed of three channels of RGB, and may have a different image size (number of pixels in the vertical and horizontal directions) depending on the size of the pathological specimen.

FIG. 2 shows an example of the pathological image. The pathological image includes a tissue region in which the pathological specimen is imaged and a background region in which a slide glass on which the pathological specimen is not arranged is imaged. In the absence of a lesion such as cancer, the tissue region is composed of a normal region that does not include a lesion image. This pathological image is divided into 12 in the horizontal direction and the vertical direction, and is divided into a total of 144 local regions.

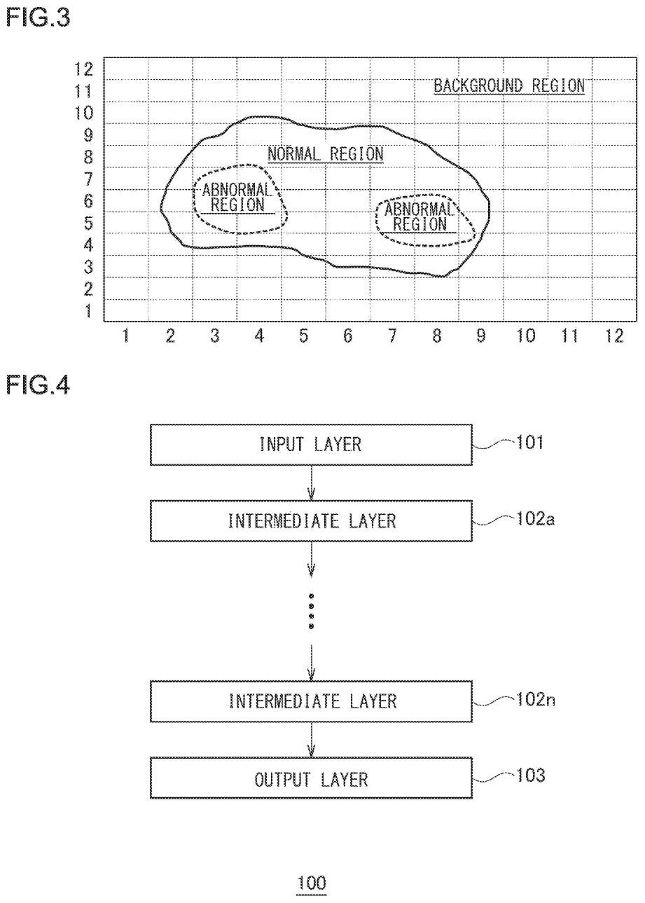

FIG. 3 shows an example of a pathological image containing abnormal regions. When a tissue contains a lesion, the tissue region is composed of a normal region containing no lesion and an abnormal region containing the lesion. In FIG. 3, abnormal regions are shown as continuous regions having a certain size; however, the size of the regions varies and may be the size of one cell.

The image analysis device 20 divides the input image into a plurality of local regions and calculates a category for each of the plurality of local regions. A local region is composed of one or more consecutive pixels. In the embodiment, the category of each local region is set to any one of a category indicating normality (normal region category), a category indicating lesion (abnormal region category), and a category indicating background region (background region category).

The image analysis device 20 performs image analysis on the local region and the surrounding region thereof, and calculates the probability of each category of the local region (probability of being estimated to be in the category of the local region). In FIG. 3, the horizontal axis is set to be the X axis, the vertical axis is set to be the Y axis, and the position of the local region is represented by (X coordinate, Y coordinate). Hereinafter, the probability of categories calculated in some local regions will be illustrated.

Probability of each category in a local region of (7,5)
normal region category: 20%
abnormal region category: 70%
background region category: 10%

Probability of each category in a local region of (4,10)
normal region category: 30%
abnormal region category: 10%
background region category: 60%

Probability of each category in a local region of (7,8)
normal region category: 46%
abnormal region category: 44%
background region category: 10%

As will be described later, the image analysis device 20 executes a probability change process of adjusting the calculated probability of each category according to the purpose. The probability change process is a process of computing and then changing the calculated probability. The probability change process may include: adding a predetermined value to the calculated probability, subtracting a predetermined value from the calculated probability, multiplying the calculated probability by a predetermined value, and/or dividing the calculated probability by a predetermined value. The probability change process may be executed for the probabilities of all the categories. Since the image analysis device 20 according to the embodiment sets the abnormal region category as a detection target category, the probability change process may be executed only for the probability of the abnormal region category.

After executing the probability change process, the image analysis device 20 identifies a category with the highest probability from among the probability of a normal region category, the probability of an abnormal region category, and the probability of a background region category for each local region and calculates the identified category as the category of the local region.

Upon calculating the category of each local region, the image analysis device 20 calculates the category (image category) of the entire image from the calculated category of each local region. The image category is either a category indicating that the image is normal (normal image category) or a category indicating that the image is abnormal (abnormal image category). In the embodiment, if even one abnormal region category is included in the plurality of local region categories, the category of the input image is calculated as an abnormal image category. On the other hand, if no abnormal region category is included in the plurality of local region categories, the category of the input image is calculated as a normal image category.

The image analysis device 20 according to the embodiment calculates the local region categories by at least two systems. A first local region category calculated by the first system is used to calculate the category (image category) of the entire image. A second local region category calculated by the second system is used for output to a user such as a pathologist. The second local region category may be output as a colored image in the local region of the abnormal region category. The image analysis device 20 outputs the image category and the second local region category to the display device 40, and the display device 40 displays the image category and the second local region category on the screen of the display device 40.

The image analysis device 20 has a convolutional neural network (CNN) that analyzes an image of a local region. FIG. 4 shows the configuration of a CNN 100. The CNN 100 includes an input layer 101, a plurality of intermediate layers 102a to 102n (hereinafter, referred to as “intermediate layer 102” when not particularly distinguished), and an output layer 103. The intermediate layer 102 includes a convolution layer and a pooling layer and improves the analysis accuracy of an input image by learning a weight and a bias, which are the parameters of the node of the convolution layer. The analysis accuracy required for the CNN 100 in the embodiment is the accuracy for accurately categorizing a local region.

In the CNN 100, a plurality of nodes in each layer are connected to a plurality of nodes in the subsequent layer. Each node outputs the sum obtained by adding the bias after obtaining the vector product of the input value from the previous layer and the weight to the node of the subsequent layer after processing the sum with a predetermined activation function. The weight and bias of each node are changed by a learning algorithm known as backpropagation. In backpropagation, the value is propagated from the back to the front of the CNN 100 while correcting the weight and bias. The corrected amount for each weight and bias is treated as a contribution to the error and calculated by the steepest descent method, and the value of the error function is minimized by changing the weight and bias values.

In the CNN 100, the weight and bias of each node are optimally set by supervised learning using a large number of pathological images. The image analysis device 20 performs an image analysis process using a CNN 100 having learned fixed parameters. In the embodiment, the CNN 100 has a function of calculating a local region category or a function of calculating intermediate information for calculating a local region category.

Hereinafter, exemplary embodiments of the image analysis device 20 will be described with reference to the figures. Constituting elements represented by the same reference numerals in a plurality of figures realize the same or similar functions and operations.

### First Exemplary Embodiment

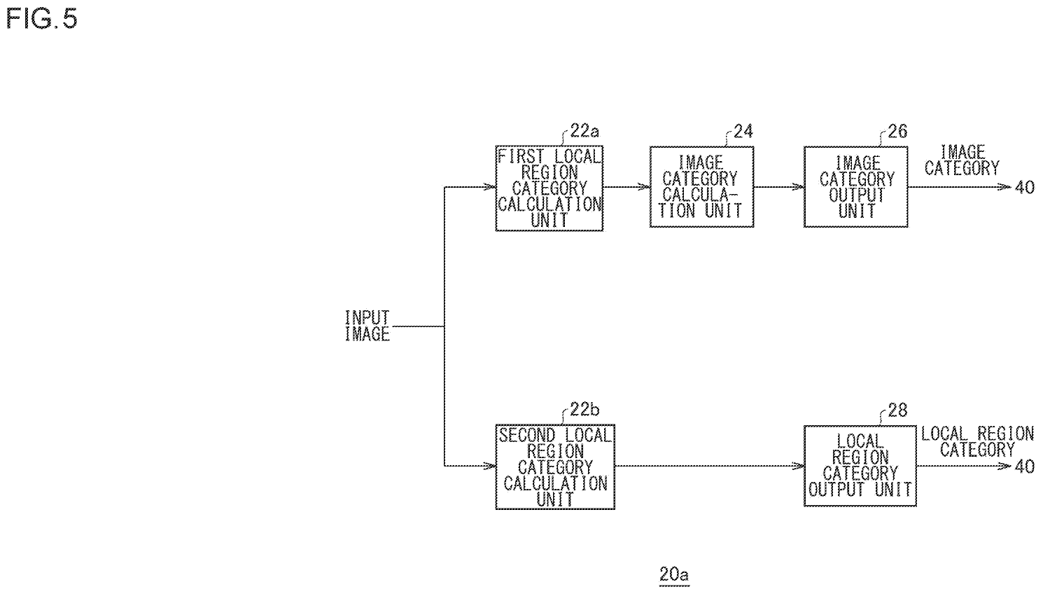

FIG. 5 shows the configuration of an image analysis device 20a according to the first exemplary embodiment. The image analysis device 20a includes a first local region category calculation unit 22a, an image category calculation unit 24, an image category output unit 26, a second local region category calculation unit 22b, and a local region category output unit 28. The first local region category calculation unit 22a calculates the categories of a plurality of local regions of an input image in the first system, and the second local region category calculation unit 22b calculates the categories of a plurality of local regions of the same input image in the second system. The first local region category calculation unit 22a and the second local region category calculation unit 22b may be configured to include a CNN 100 to which learned parameters are set.

The first local region category calculation unit 22a calculates the first local region category from the input image. The types of local region categories are a normal region category, an abnormal region category, and a background region category. The first local region category calculation unit 22a performs image analysis of a local region, calculates the probability of each category of the local region, and executes a probability change process for adjusting the calculated probability of each category. The first local region category calculation unit 22a may adjust only the probability of the abnormal region category. The first local region category calculation unit 22a compares the probability of each category that has undergone the probability change process for each local region, and calculates the category with the highest probability as the category of the local region.

The image category calculation unit 24 calculates an image category from a plurality of first local region categories calculated by the first local region category calculation unit 22a. The image category calculation unit 24 according to the first exemplary embodiment refers to all the local region categories included in the image, calculates the abnormal image category if there is even one abnormal region category, and calculates the normal image category if there is not even one abnormal region category. The image category output unit 26 outputs the image category calculated by the image category calculation unit 24 to the display device 40 and the like. The image category output unit 26 may output the calculated image category to a predetermined storage device in order to associate the image category with the input image. In the storage device, the image category is stored in association with the input image. In the image analysis device 20a, the first local region category is used for calculating the image category and is not output for display. As will be described later, in the image analysis device 20a, the second local region category described later is output from the display device 40 as the local region category. As a result, the image analysis device 20a outputs a category of a local region useful for image observation by the user while calculating the image category with high accuracy.

The second local region category calculation unit 22b calculates the second local region category from the same input image. The types of local region categories are a normal region category, an abnormal region category, and a background region category. The second local region category calculation unit 22b performs image analysis of a local region, calculates the probability of each category of the local region, and executes a probability change process for adjusting the calculated probability of each category. The second local region category calculation unit 22b may adjust only the probability of the abnormal region category. The second local region category calculation unit 22b compares the probability of each category that has undergone the probability change process for each local region, and calculates the category with the highest probability as the category of the local region.

The local region category output unit 28 outputs the second local region category calculated by the second local region category calculation unit 22b to the display device 40. The local region category output unit 28 may output the second local region category in various modes. As one output mode, the local region category output unit 28 may generate and output a pathological image that has undergone a visualization process such as coloring the position of a local region classified into the abnormal region category in the image. Visualization of local regions classified into the abnormal region category is expected to improve the observation efficiency by pathologists.

In the first local region category calculation unit 22a and the second local region category calculation unit 22b, the calculation of the probability of each category before the probability change process may be executed using the CNN 100 to which the same learned parameters are set. Therefore, the calculated probability of each category is the same in both the first local region category calculation unit 22a and the second local region category calculation unit 22b. In the first exemplary embodiment, the first local region category calculation unit 22a and the second local region category calculation unit 22b execute different probability change processes for the same calculated category probability due to the difference in the purpose of use of the first local region category and the second local region category.

In order to realize highly accurate calculation of the image category by the image category calculation unit 24, the first local region category calculation unit 22a executes the probability change process so as to optimize each category probability for the image category calculation. In the case of a pathological image, there are many structures in a normal local region that have characteristics similar to abnormalities. Therefore, in an algorithm by which the entire image is determined to be an abnormal image if there is even one abnormal region category, a normal local region is easily classified as an abnormal region category erroneously, and an image not containing abnormalities is easily determined to be an abnormal image erroneously. Therefore, the first local region category calculation unit 22a reduces the possibility of erroneously determining an image not containing abnormalities to be an abnormal image by executing a probability change process that lowers the probability value of an abnormal region category.

The first local region category calculation unit 22a lowers the probability value of the abnormal region category by, for example, 20%. According to this probability change process, the respective probabilities of the categories at (7,5), (4,10), and (7,8) calculated by the first local region category calculation unit 22a are as follows.

Probability of each category in a local region of (7,5)
normal region category: 20%
abnormal region category: 50% (=70%-20%)
background region category: 10%

Probability of each category in a local region of (4,10)
normal region category: 30%
abnormal region category: −10% (=10%-20%)
background region category: 60%

Probability of each category in a local region of (7,8)
normal region category: 46%
abnormal region category: 24% (=44%-20%)
background region category: 10%

Therefore, the first local region category calculation unit 22a calculates the first local region category as follows.
local region category at (7,5): abnormal region category
local region category at (4,10): background region category
local region category at (7,8): normal region category

The second local region category calculation unit 22b executes the probability change process so as to optimize each category probability used for the purpose of presentation to the user. The second local region category calculated by the second local region category calculation unit 22b is used for performing a marking process on the local region classified as the abnormal region category. Therefore, for example, a local region in which the probability of the abnormal region category and the probability of the normal region category are almost the same is preferably classified into the abnormal region category in a forced manner and carefully observed by a pathologist.

Therefore, when the probability change process of the first local region category calculation unit 22a and the probability change process of the second local region category calculation unit 22b are compared, the percentage of detection target categories (abnormal region categories) occupying a plurality of second local region categories calculated by the second local region category calculation unit 22b is equal to or greater than the percentage of detection target categories (abnormal region categories) occupying a plurality of first local region categories calculated by the first local region category calculation unit 22a. That is, the first set of local regions calculated to be in the abnormal region category by the first local region category calculation unit 22a is a subset of the second set of local regions calculated to be in the abnormal region category by the second local region category calculation unit 22b.

The second local region category calculation unit 22b raises the probability value of the abnormal region category by, for example, 10%. According to this probability change process, the respective probabilities of the categories at (7,5), (4,10), and (7,8) calculated by the second local region category calculation unit 22b are as follows.

Probability of each category in a local region of (7,5)
normal region category: 20%
abnormal region category: 80% (=70%+10%)
background region category: 10%

Probability of each category in a local region of (4,10)
normal region category: 30%
abnormal region category: 20% (=10%+10%)
background region category: 60%

Probability of each category in a local region of (7,8)
normal region category: 46%
abnormal region category: 54% (=44%+10%)
background region category: 10%

Therefore, the second local region category calculation unit 22b calculates the second local region category as follows.
local region category at (7,5): abnormal region category
local region category at (4,10): background region category
local region category at (7,8): abnormal region category

Compared with the local region category calculated by the first local region category calculation unit 22a, the calculation result of the local region category at (7,8), calculated by the second local region category calculation unit 22b, is different. That is, while the first local region category at (7,8) is a normal region category, the second local region category is calculated as an abnormal region category.

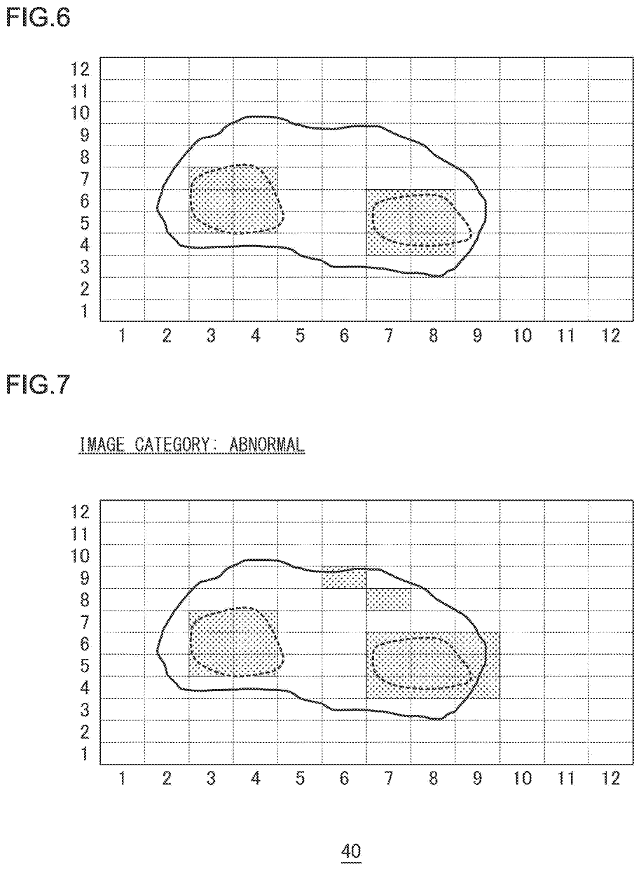

FIG. 6 shows an output example of the first local region category calculated by the first local region category calculation unit 22a. The output example of the first local region category shown in FIG. 6 is for comparison with an output example of the second local region category shown in FIG. 7, and in the image analysis device 20 of the embodiment, the first local region category is not output.

The first local region category calculation unit 22a classifies local regions at (3,5), (3,6), (3,7), (4,5), (4,6), (4,7), (7,4), (7,5), (7,6), (8,4), (8,5), and (8,6) into the abnormal region category.

FIG. 7 shows the output of the image category by the image category output unit 26 and an output example of the second local region category calculated by the second local region category calculation unit 22b. The display device 40 displays the image category as text. And the display device 40 displays local regions of the abnormal region category which are colored through the marking process.

The second local region category calculation unit 22b classifies local regions at (3,5), (3,6), (3,7), (4,5), (4,6), (4,7), (6,9), (7,4), (7,5), (7,6), (7,8), (8,4), (8,5), (8,6), (9,4), (9,5), and (9,6) into the abnormal region category.

Comparing FIGS. 6 and 7, the second local region category calculation unit 22b classifies local regions at (6,9), (7,8), (9,4), (9,5), and (9,6) into the abnormal region category in addition to the local regions of the abnormal region category calculated by the first local region category calculation unit 22a. That is, in the second local region category, more local regions are classified into the abnormal region category. Even when whether a region is a normal region or an abnormal region cannot be perfectly determined by the CNN 100, by marking the region and presenting the marked region to the pathologist, the pathologist can carefully observe the marked local region. Thus, efficient diagnosis by the pathologist can be supported.

In the first exemplary embodiment, an explanation is given stating that the calculation of the probability of each category may be executed using the CNN 100 to which the same learned parameters are set in the first local region category calculation unit 22a and the second local region category calculation unit 22b. However, the size of the local region that determines the first local region category and the size of the local region that determines the second local region category may be different.

For example, the resolution of the second local region category calculated by the second local region category calculation unit 22b may be lower than the resolution of the first local region category calculated by the first local region category calculation unit 22a. In the second local region category calculation unit 22b, the stride of the CNN 100 may be doubled as compared to that of in the first local region category calculation unit 22a, or the resolution of the output data of the CNN 100 may be converted. For example, the number of local regions divided by the second local region category calculation unit 22b may be ¼ times the number of local regions divided by the first local region category calculation unit 22a.

In the image analysis device 20a, the local region category output unit 28 may generate image data in which the positions of the local regions of the abnormal region category are marked and output the image data to the display device 40. However, the generation of the image data may be executed in another processing unit. In this case, the local region category output unit 28 may output the second local region category to the processing unit, and the processing unit may generate the image data and output the image data to the display device 40. Further, the local region category output unit 28 may output the calculated second local region category to a predetermined storage device in order to associate the image category with the input image. In the storage device, the image category and the second local region category output from the image analysis device 20a are stored in association with the input image.

According to the image analysis device 20a according to the first exemplary embodiment, it is possible to estimate an image category with high accuracy and calculate a local region category useful for image observation by the user by calculating a local region category in two systems.

### Second Exemplary Embodiment

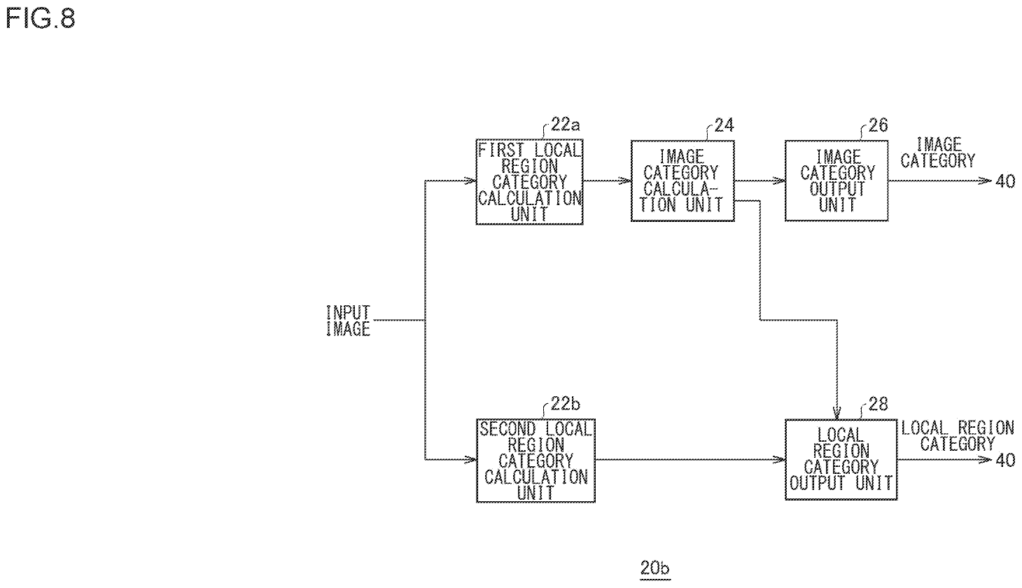

FIG. 8 shows the configuration of an image analysis device 20b according to the second exemplary embodiment. The image analysis device 20b includes a first local region category calculation unit 22a, an image category calculation unit 24, an image category output unit 26, a second local region category calculation unit 22b, and a local region category output unit 28. The first local region category calculation unit 22a calculates the categories of a plurality of local regions of an input image in the first system, and the second local region category calculation unit 22b calculates the categories of a plurality of local regions of the same input image in the second system. The first local region category calculation unit 22a and the second local region category calculation unit 22b may be configured to include a CNN 100 to which learned parameters are set.

Compared with the image analysis device 20a according to the first exemplary embodiment, in the image analysis device 20b according to the second exemplary embodiment, the image category calculated by the image category calculation unit 24 is supplied to the local region category output unit 28 as control information for the local region category output unit 28. The calculated image category is either a normal image category or an abnormal image category, and when the image category calculation unit 24 calculates the image category, the image category is supplied to the local region category output unit 28.

When the image category is an abnormal image category, the local region category output unit 28 outputs the second local region category. The second local region category may be output as pathological image data in which the position of the local region of the abnormal region category is visualized.

On the other hand, when the image category is a normal image category, the local region category output unit 28 does not output the second local region category. The normal image category indicates that the image does not contain any abnormality. However, if the local region category output from the local region category output unit 28 includes the abnormal region category, the image analysis result does not become consistent. Therefore, in the image analysis device 20b, when the image category is the normal image category, the local region category output unit 28 does not output the second local region category, thereby avoiding a situation in which the inconsistent analysis result is presented to the user.

### Third Exemplary Embodiment

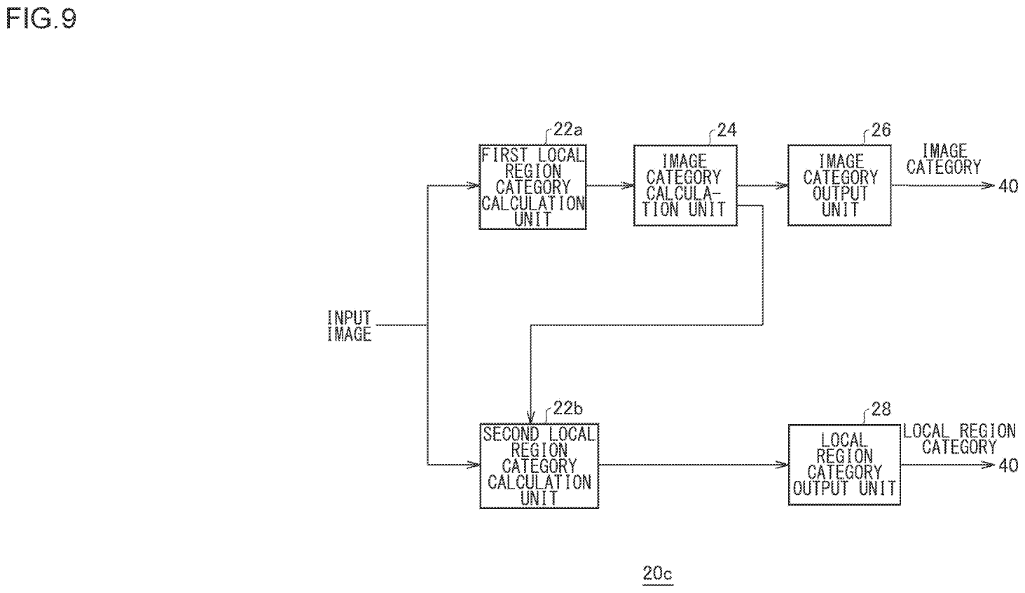

FIG. 9 shows the configuration of an image analysis device 20c according to the third exemplary embodiment. The image analysis device 20c includes a first local region category calculation unit 22a, an image category calculation unit 24, an image category output unit 26, a second local region category calculation unit 22b, and a local region category output unit 28. The first local region category calculation unit 22a calculates the categories of a plurality of local regions of an input image in the first system, and the second local region category calculation unit 22b calculates the categories of a plurality of local regions of the same input image in the second system. The first local region category calculation unit 22a and the second local region category calculation unit 22b may be configured to include a CNN 100 to which learned parameters are set.

Compared with the image analysis device 20a according to the first exemplary embodiment, in the image analysis device 20c according to the third exemplary embodiment, the image category calculated by the image category calculation unit 24 is supplied to the second local region category calculation unit 22b as control information for the second local region category calculation unit 22b. The calculated image category is either a normal image category or an abnormal image category, and when the image category calculation unit 24 calculates the image category, the image category is supplied to the second local region category calculation unit 22b.

When the image category is an abnormal image category, the second local region category calculation unit 22b calculates the second local region category. The calculated second local region category is supplied to the local region category output unit 28 and is output to the display device 40 along with the image category.

On the other hand, when the image category is a normal image category, the second local region category calculation unit 22b does not calculate the second local region category. The normal image category indicates that the image does not contain any abnormality. However, if the local region category calculated by the second local region category calculation unit 22b includes the abnormal region category, the image analysis result does not become consistent. Therefore, when the image category is a normal image category, by not allowing the second local region category calculation unit 22b to calculate the second local region category, a situation in which the inconsistent analysis result is presented to the user is avoided, and an arithmetic process by the second local region category calculation unit 22b is stopped.

### Fourth Exemplary Embodiment

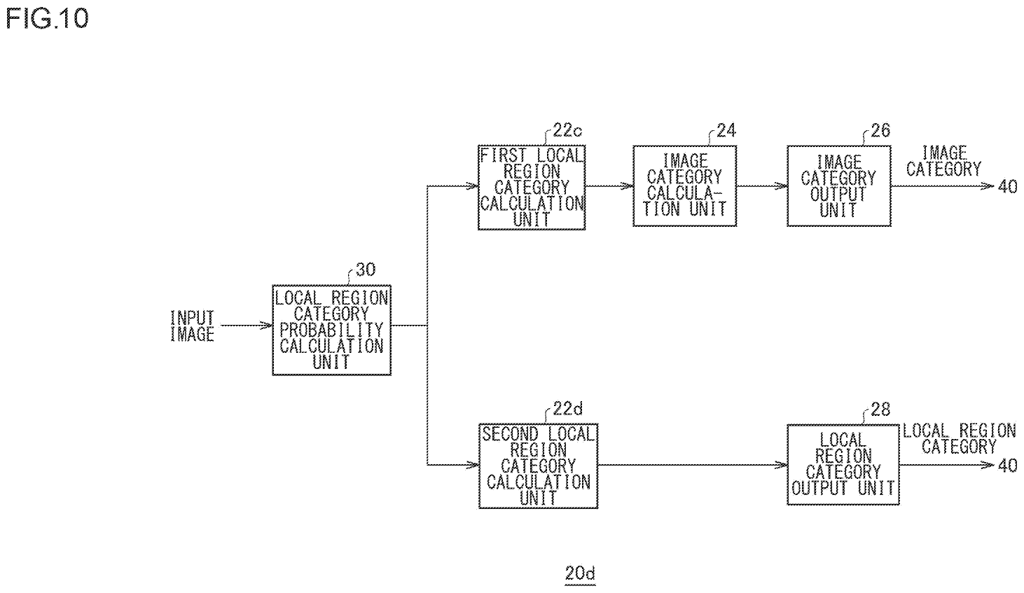

FIG. 10 shows the configuration of an image analysis device 20d according to the fourth exemplary embodiment. The image analysis device 20d includes a local region category probability calculation unit 30, a first local region category calculation unit 22c, an image category calculation unit 24, an image category output unit 26, a second local region category calculation unit 22d, and a local region category output unit 28. The first local region category calculation unit 22c calculates the categories of a plurality of local regions of an input image in the first system, and the second local region category calculation unit 22d calculates the categories of a plurality of local regions of the same input image in the second system.

The local region category probability calculation unit 30 according to the fourth exemplary embodiment performs the calculation process of each category probability before the probability change process in the first exemplary embodiment. In the first exemplary embodiment, an explanation is made stating that the first local region category calculation unit 22a and the second local region category calculation unit 22b execute the calculation of the probability of each category before the probability change process by using the CNN 100. The local region category probability calculation unit 30 according to the fourth exemplary embodiment uses CNN 100 to calculate the probability of each category before the probability change process.

In the first exemplary embodiment, the first local region category calculation unit 22a and the second local region category calculation unit 22b calculate the probability of each category in a duplicate manner. However, in the fourth exemplary embodiment, the local region category probability calculation unit 30 representatively calculates the probability of each category and supplies thus calculated probability of each category to both the first local region category calculation unit 22c and the second local region category calculation unit 22d. As a result, the calculation process of each category probability, which is performed in a duplicate manner in the first exemplary embodiment, can be performed only once.

The first local region category calculation unit 22c executes the probability change process on the supplied probability value of each category, compares the probability of each category on which the probability change process has been performed, and calculates the category with the highest probability as the first local region category. In the same manner, the second local region category calculation unit 22d executes the probability change process on the supplied probability value of each category, compares the probability of each category on which the probability change process has been performed, and calculates the category with the highest probability as the second local region category. The probability change process by the first local region category calculation unit 22c and the probability change process by the second local region category calculation unit 22d are the same as the probability change process by the first local region category calculation unit 22a and the probability change process by the second local region category calculation unit 22b in the first exemplary embodiment. The operation of the image category calculation unit 24, the image category output unit 26, and the local region category output unit 28 are the same as the operation of the image category calculation unit 24, the image category output unit 26, and the local region category output unit 28 according to the first exemplary embodiment.

According to the image analysis device 20d of the fourth exemplary embodiment, the calculation of the category probability in a local region by the local region category probability calculation unit 30 allows the duplicate category probability calculation process to be avoided.

### Fifth Exemplary Embodiment

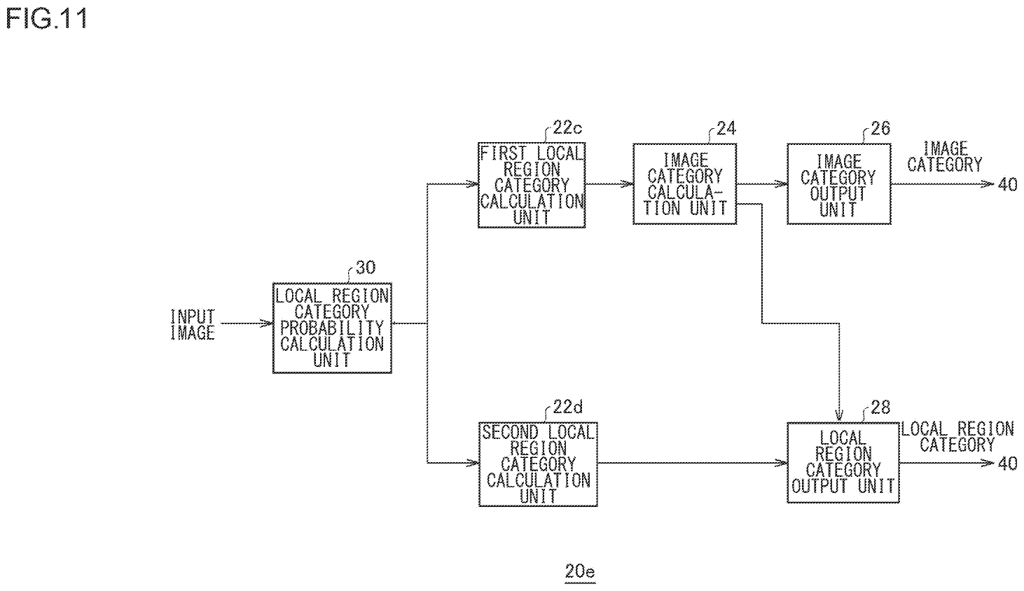

FIG. 11 shows the configuration of an image analysis device 20e according to the fifth exemplary embodiment. The image analysis device 20e includes a local region category probability calculation unit 30, a first local region category calculation unit 22c, an image category calculation unit 24, an image category output unit 26, a second local region category calculation unit 22d, and a local region category output unit 28. The first local region category calculation unit 22c calculates the categories of a plurality of local regions of an input image in the first system, and the second local region category calculation unit 22d calculates the categories of a plurality of local regions of the same input image in the second system.

Compared with the image analysis device 20d according to the fourth exemplary embodiment, in the image analysis device 20e according to the fifth exemplary embodiment, the image category calculated by the image category calculation unit 24 is supplied to the local region category output unit 28 as control information for the local region category output unit 28. The calculated image category is either a normal image category or an abnormal image category, and when the image category calculation unit 24 calculates the image category, the image category is supplied to the local region category output unit 28.

When the image category is an abnormal image category, the local region category output unit 28 outputs the second local region category. The second local region category may be output as pathological image data in which the position of the local region of the abnormal region category is visualized.

On the other hand, when the image category is a normal image category, the local region category output unit 28 does not output the second local region category. The normal image category indicates that the image does not contain any abnormality. However, if the local region category output from the local region category output unit 28 includes the abnormal region category, the image analysis result does not become consistent. Therefore, in the image analysis device 20e, when the image category is the normal image category, the local region category output unit 28 does not output the second local region category, thereby avoiding a situation in which the inconsistent analysis result is presented to the user.

### Sixth Exemplary Embodiment

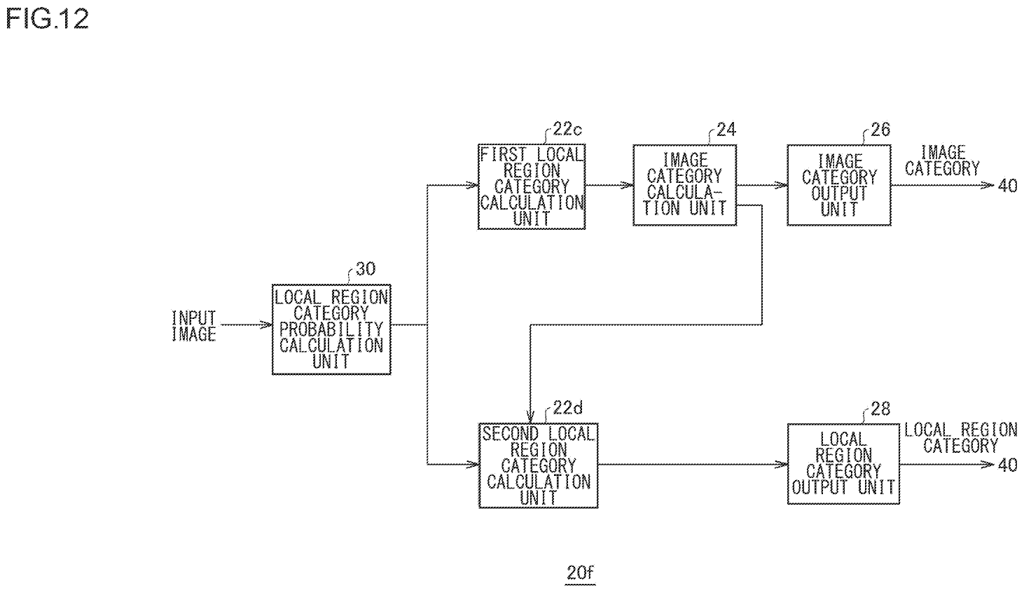

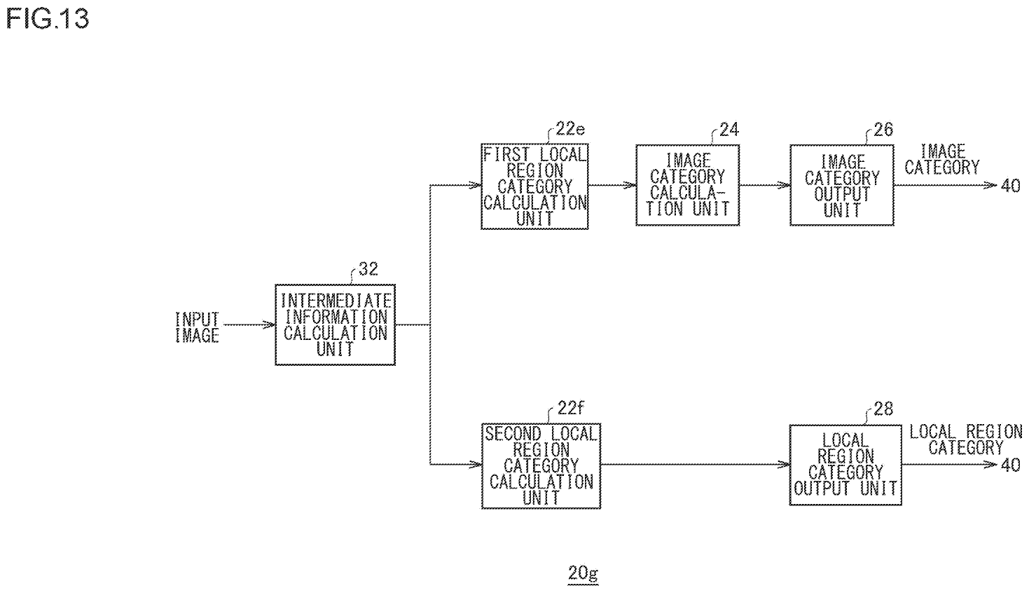

FIG. 12 shows the configuration of an image analysis device 20f according to the sixth exemplary embodiment. The image analysis device 20f includes a local region category probability calculation unit 30, a first local region category calculation unit 22c, an image category calculation unit 24, an image category output unit 26, a second local region category calculation unit 22d, and a local region category output unit 28. The first local region category calculation unit 22c calculates the categories of a plurality of local regions of an input image in the first system, and the second local region category calculation unit 22d calculates the categories of a plurality of local regions of the same input image in the second system.

Compared with the image analysis device 20d according to the fourth exemplary embodiment, in the image analysis device 20f according to the sixth exemplary embodiment, the image category calculated by the image category calculation unit 24 is supplied to the second local region category calculation unit 22d as control information for the second local region category calculation unit 22d. The calculated image category is either a normal image category or an abnormal image category, and when the image category calculation unit 24 calculates the image category, the image category is supplied to the second local region category calculation unit 22d.

When the image category is an abnormal image category, the second local region category calculation unit 22d calculates the second local region category. The calculated second local region category is supplied to the local region category output unit 28 and is output to the display device 40 along with the image category.

On the other hand, when the image category is a normal image category, the second local region category calculation unit 22d does not calculate the second local region category. The normal image category indicates that the image does not contain any abnormality. However, if the local region category calculated by the second local region category calculation unit 22d includes the abnormal region category, the image analysis result does not become consistent. Therefore, when the image category is a normal image category, by not allowing the second local region category calculation unit 22d to calculate the second local region category, a situation in which the inconsistent analysis result is presented to the user is avoided, and an arithmetic process by the second local region category calculation unit 22d is stopped.

### Seventh Exemplary Embodiment

FIG. 13 shows the configuration of an image analysis device 20g according to the seventh embodiment. The image analysis device 20g includes an intermediate information calculation unit 32, a first local region category calculation unit 22e, an image category calculation unit 24, an image category output unit 26, a second local region category calculation unit 22f, and a local region category output unit 28. The first local region category calculation unit 22e calculates the categories of a plurality of local regions of an input image in the first system, and the second local region category calculation unit 22f calculates the categories of a plurality of local regions of the same input image in the second system.

The intermediate information calculation unit 32 according to the seventh exemplary embodiment calculates intermediate information for calculating a local region category from the input image for each local region, and supplies the calculated intermediate information to both the first local region category calculation unit 22c and the second local region category calculation unit 22d. The intermediate information calculation unit 32 according to the seventh exemplary embodiment may calculate the intermediate information by using the CNN 100. The intermediate information may be the image feature value of the local region or the probability of each of the plurality of categories of the local region.

When the intermediate information is the image feature value of the local region, the first local region category calculation unit 22e calculates the probability of each of the plurality of categories of the local region from the intermediate information, executes the probability change process on the probability value of each category, compares the probability of each category on which the probability change process has been performed, and calculates the category with the highest probability as the first local region category. In the same manner, the second local region category calculation unit 22f calculates the probability of each of the plurality of categories of the local region from the intermediate information, executes the probability change process on the probability value of each category, compares the probability of each category on which the probability change process has been performed, and calculates the category with the highest probability as the first local region category. The probability change process by the first local region category calculation unit 22e and the probability change process by the second local region category calculation unit 22f are the same as the probability change process by the first local region category calculation unit 22a and the probability change process by the second local region category calculation unit 22b in the first exemplary embodiment. The operation of the image category calculation unit 24, the image category output unit 26, and the local region category output unit 28 are the same as the operation of the image category calculation unit 24, the image category output unit 26, and the local region category output unit 28 according to the first exemplary embodiment.

### Eighth Exemplary Embodiment

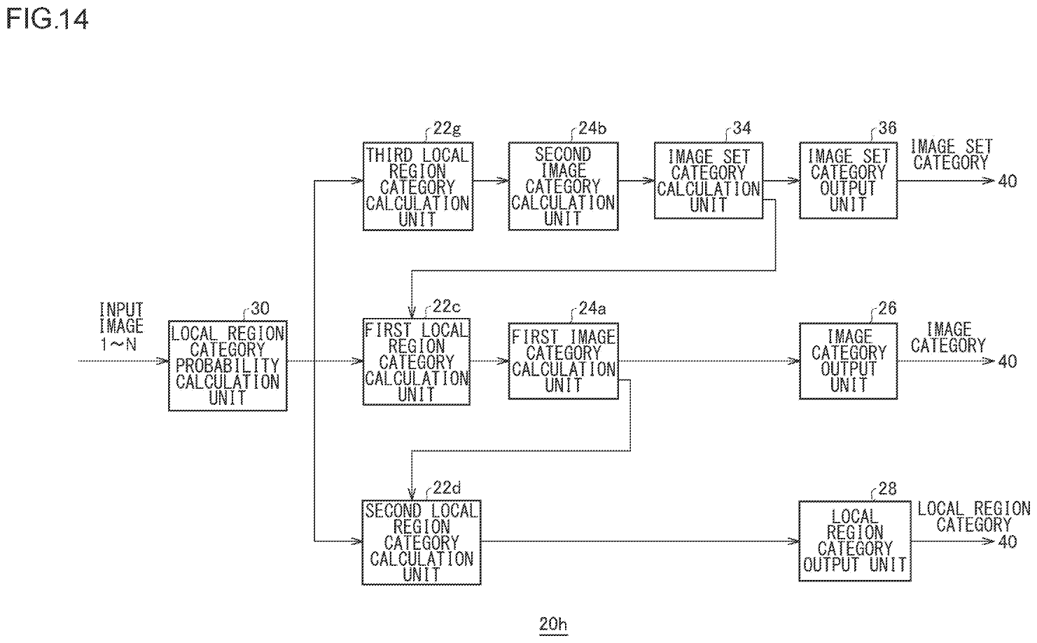

FIG. 14 shows the configuration of an image analysis device 20h according to the eighth exemplary embodiment. The image analysis device 20h includes a local region category probability calculation unit 30, a first local region category calculation unit 22c, a first image category calculation unit 24a, an image category output unit 26, a second local region category calculation unit 22d, a local region category output unit 28, a third local region category calculation unit 22g, a second image category calculation unit 24b, an image set category calculation unit 34, and an image set category output unit 36. The local region category probability calculation unit 30, the first local region category calculation unit 22c, the first image category calculation unit 24a, the image category output unit 26, the second local region category calculation unit 22d, and the local region category output unit 28 in the image analysis device 20h according to the eighth exemplary embodiment correspond to the local region category probability calculation unit 30, the first local region category calculation unit 22c, the image category calculation unit 24, the image category output unit 26, the second local region category calculation unit 22d, and the local region category output unit 28 in the image analysis device 20f according to the sixth exemplary embodiment.

In the image analysis system 1, the image supply unit 10 inputs N (N is one or more) pathological images of a tissue section acquired from one specimen (patient) into the image analysis device 20h as one image set. The image analysis device 20h outputs an image set category indicating whether the image set is normal or abnormal, outputs N image categories indicating whether each of the N images are normal or abnormal when the image set is abnormal, and further outputs, with regard to an image with an abnormal image category, a local region category indicating whether the local region of the image is abnormal or not.

Upon the input of the N input images, the local region category probability calculation unit 30 calculates N local region category probabilities corresponding to the plurality of input images, respectively. The process of the local region category probability calculation unit 30 on one input image is the same as the process of the local region category probability calculation unit 30 in the image analysis device 20d according to the fourth exemplary embodiment shown in FIG. 10. The local region category probability calculation unit 30 according to the eighth exemplary embodiment calculates the probability of each category before the probability change process by using the CNN 100. The local region category probability calculation unit 30 of the image analysis device 20h calculates the local region category probability of each of the N input images.

The third local region category calculation unit 22g calculates the third local region category corresponding to each of the plurality of input images. More specifically, the third local region category calculation unit 22g executes the probability change process on the supplied probability value of each category, compares the probability of each category on which the probability change process has been performed, and calculates the category with the highest probability as the third local region category.

From the plurality of third local region categories of each input image calculated by the third local region category calculation unit 22g, the second image category calculation unit 24b calculates an image category that corresponds to each of the third local region categories. The second image category calculation unit 24b according to the eighth exemplary embodiment refers to all the local region categories included in the image, calculates the abnormal image category if there is even one abnormal region category, and calculates the normal image category if there is not even one abnormal region category.

The image set category calculation unit 34 calculates the image set category from a plurality of image categories. More specifically, upon the input of the N input images, the image set category calculation unit 34 determines that the image set category is abnormal if, out of all the image categories, even one abnormal image category is included, determines that the image category is normal if not even one abnormal image category is included, and outputs the image set category.

In the eighth exemplary embodiment, the first local region category calculation unit 22c, the second local region category calculation unit 22d, and the third local region category calculation unit 22g execute different probability change processes for the category probability calculated by the local region category probability calculation unit 30 due to the differences in the purpose of use of the first local region category, the second local region category, and the third local region category. The probability change process by the first local region category calculation unit 22c and the probability change process by the second local region category calculation unit 22d may be the same as the probability change process by the first local region category calculation unit 22a and the probability change process by the second local region category calculation unit 22b in the first exemplary embodiment.

The third local region category is used to calculate the image set category indicating whether the image set is normal or abnormal. Whether the image set is normal or abnormal is synonymous with whether the patient does not have or has a lesion. The third local region category calculation unit 22g executes the probability change process to optimize each category probability for image set category calculation. The third local region category calculation unit 22g reduces the possibility of erroneously determining an image set including no abnormality to be abnormal by executing a probability change process that lowers the probability value of the abnormal region category.

When the probability change process of the third local region category calculation unit 22g and the probability change process of the first local region category calculation unit 22c are compared, the percentage of detection target categories (abnormal region categories) occupying a plurality of first local region categories calculated by the first local region category calculation unit 22c is equal to or greater than the percentage of detection target categories (abnormal region categories) occupying a plurality of third local region categories calculated by the third local region category calculation unit 22g. That is, the third set of local regions calculated to be in the abnormal region category by the third local region category calculation unit 22g is a subset of the first set of local regions calculated to be in the abnormal region category by the first local region category calculation unit 22c.

The third local region category calculation unit 22g lowers the probability value of the abnormal region category by, for example, 30%. According to this probability change process, the respective probabilities of the categories at (7,5), (4,10), and (7,8) calculated by the third local region category calculation unit 22g are as follows.

Probability of each category in a local region of (7,5)
normal region category: 20%
abnormal region category: 40% (=70%-30%)
background region category: 10%

Probability of each category in a local region of (4,10)
normal region category: 30%
abnormal region category: −20% (=10%-30%)
background region category: 60%

Probability of each category in a local region of (7,8)
normal region category: 46%
abnormal region category: 14% (=44%-30%)
background region category: 10%

Therefore, the first local region category calculation unit 22a calculates the first local region category as follows.
local region category at (7,5): abnormal region category
local region category at (4,10): background region category
local region category at (7,8): normal region category

The image set category calculation unit 34 calculates an image set category expressing whether the image set of one patient is normal or abnormal, and when the image set category shows an abnormality, the first local region category calculation unit 22c calculates the first local region category of N input images. When the first image category calculation unit 24a calculates each image category of each input image, the second local region category calculation unit 22d calculates the category of a local region for the image whose image category shows an abnormality. The image set category output unit 36 outputs the image set category, the image category output unit 26 outputs the image category, and the local region category output unit 28 outputs the local region category. According to the image analysis device 20h according to the eighth exemplary embodiment, an image set of one patient can be efficiently analyzed.

Described above is an explanation on the present disclosure based on the embodiments and the exemplary embodiments. These exemplary embodiments are intended to be illustrative only, and it will be obvious to those skilled in the art that various modifications to constituting elements and processes could be developed and that such modifications are also within the scope of the present disclosure.

In the exemplary embodiments, the first local region category and the second local region category are both determined as one of the categories included in the common group. That is, in the exemplary embodiments, the first local region category and the second local region category are determined from a local region category group including three categories: a normal region category; an abnormal region category; and a background region category.

In an exemplary variation, the first group and the second group of a local region category may be prepared, the first local region category may be determined as one of categories included in the first group, and the second local region category may be determined as one of categories included in the second group. A category group that constitutes the first group and a category group that constitutes the second group are different.

For example, the first local region category includes subcategories in which the abnormal region is subdivided. When the abnormal region includes an image region of a lesion belonging to gastric cancer, the abnormal region may be subdivided into three categories: a highly-differentiated tubular adenocarcinoma category, a moderately-differentiated tubular adenocarcinoma category, and a poorly-differentiated adenocarcinoma category. That is, for each local region, the first local region category calculation units 22a, 22c, and 22e calculate the first local region category classified into any one of the highly-differentiated tubular adenocarcinoma category, the moderately-differentiated tubular adenocarcinoma category, the poorly-differentiated adenocarcinoma category, and the background region category. In an exemplary variation, the image category calculation unit 24 treats the highly-differentiated tubular adenocarcinoma category, the moderately-differentiated tubular adenocarcinoma category, and the poorly-differentiated adenocarcinoma category as abnormal region categories. In this exemplary variation, there is an effect that the first local region category can be analyzed with high accuracy by classifying the abnormality of the first local region category into subdivided subcategories. At this time, the second local region category does not include subcategories, and the same category group as those according to the exemplary embodiments may be used.

In the embodiments, the exemplary embodiments, and the exemplary variations, an image analysis device may include a processor and storage such as memory. In the processor in this case, for example, the function of each part may be realized by individual hardware, or the function of each part may be realized by integrated hardware. For example, the processor includes hardware, and the hardware can include at least one of a circuit that processes digital signals and a circuit that processes analog signals. For example, the processor can consist of one or more circuit devices (e.g., ICs, etc.) mounted on a circuit board, or one or more circuit elements (e.g., resistors, capacitors, etc.). The processor may be, for example, a central processing unit (CPU). However, the processor is not limited to a CPU, and may include various processors such as a graphics processing unit (GPU) or a digital signal processor (DSP). Further, the processor may be a hardware circuit by an application specific integrated circuit (ASIC) or a field-programmable gate array (FPGA). Further, the processor may include an amplifier circuit, a filter circuit, and the like for processing analog signals. The memory may be a semiconductor memory such as SRAM or DRAM, a register, a magnetic storage device such as a hard disk device, or an optical storage device such as an optical disk device. For example, the memory stores instructions that can be read by a computer, and when the instructions are executed by the processor, the function of each part of the image analysis device is realized. The instructions in this case may be instructions of an instruction set constituting a program, or an instruction instructing an operation to a hardware circuit of the processor.

Further, in the embodiments, the exemplary embodiments, and the exemplary variations, each processing unit of the image analysis device may be connected by, for example, any type or medium of digital data communication such as a communication network. Examples of the communication network include, for example, LANs, WANs, and computers and networks that form the Internet.

What is claimed is:

1. An image analysis device comprising:
a processor comprising hardware, wherein the processor is configured to:
calculate a first local region category of an input image;
calculate an image category from the first local region category;
output the image category;
calculate a second local region category of the input image; and
output the second local region category.

2. The image analysis device according to claim 1, wherein
the percentage of a detection target category occupying a plurality of the second local region categories is equal to or more than the percentage of a detection target category occupying a plurality of the first local region categories.

3. The image analysis device according to claim 2, wherein
a first set of local regions calculated to be in the detection target category in the plurality of the first local region categories is a subset of a second set of local regions calculated to be in the detection target category in the plurality of the second local region categories.

4. The image analysis device according to claim 2, wherein
the input image is a pathological image, and the detection target category is a category indicating a lesion.

5. The image analysis device according to claim 1, wherein the processor is configured to:
when the image category is a first image category, calculate the second local region category; and
when the image category is a second image category, not calculate the second local region category.

6. The image analysis device according to claim 1, wherein the processor is configured to:
calculate intermediate information for calculating a local region category from the input image for each local region;
calculate the first local region category using the intermediate information; and
calculate the second local region category using the intermediate information.

7. The image analysis device according to claim 6, wherein
the intermediate information represents the image feature value of a local region or the probability of each of a plurality of categories of the local region.

8. The image analysis device according to claim 1, wherein
the first local region category is determined as one of categories included in a first group, and the second local region category is determined as one of categories included in a second group, and
the first group and the second group are different.

9. The image analysis device according to claim 1, wherein the processor is configured to:
calculate a third local region category that corresponds to each of a plurality of input images;
from a plurality of third local region categories, calculate a second image category that corresponds to each of the third local region categories; and
calculate an image set category from a plurality of second image categories.

10. The image analysis device according to claim 9, wherein
the percentage of a detection target category occupying the plurality of first local region categories is equal to or more than the percentage of a detection target category occupying the plurality of third local region categories.

11. The image analysis device according to claim 9, wherein
the plurality of input images are a plurality of pathological images acquired from a specimen of one person.

12. The image analysis device according to claim 1, wherein
the first local region category is not output.

13. The image analysis device according to claim 1, wherein the processor is configured to:
when the image category is a first image category, output the second local region category; and
when the image category is a second image category, not output the second local region category.

14. The image analysis device according to claim 1, wherein
the size of a local region that determines the first local region category and the size of a local region that determines the second local region category are different.

15. An image analysis method comprising:
calculating a first local region category of an input image;
calculating an image category from the first local region category;
outputting the image category;
calculating a second local region category of the input image; and
outputting the second local region category.

16. A recording medium having embodied thereon a program comprising computer-implemented modules including:
a module that calculates a first local region category of an input image;
a module that calculates an image category from the first local region category;
a module that outputs the image category;
a module that calculates a second local region category of the input image; and
a module that outputs the second local region category.
